(19) United States
(12) Patent Application Publication
(10) Pub. No.: US 2003/0118618 A1
(43) Pub. Date: Jun. 26, 2003

(54) New Method for making a water-in-oil emulsion

(76) Inventor: Arnold W. Fogel, Upper Saddle River, NJ (US)

Correspondence Address: Henry D. Coleman, Coleman Sudol Sapone, P.C., 714 Colorado Avenue, Bridgeport, CT, 06605-1601, US

(21) Appl. No.: 10/045,211

(22) Filed: Oct. 23, 2001

(51) Int. Cl. A61K 7/32; A61K 7/06

(52) U.S. Cl. 424/401; 424/65; 424/74

(57) ABSTRACT

The present invention relates to novel water-in-oil compositions which exhibit unexpected storage stability and other characteristics in a high water content composition. The present compositions comprise a combination of dibehenyl fumarate, PEG 1500 dihydroxystearate and monoethanolamine stearamide (also, stearic monoethanolamide) in combination with an oil and water soluble, and optionally, numerous other ingredients or components. The compositions may be used as an emulsifier mixture, or as emulsions for formulating numerous compositions, and in particular, pharmaceutical or personal care products such as cosmetics and dermatological agents. The compositions according to the present invention are particularly useful as additives in dermatological products, including cosmetic compositions. The compositions exhibit a number of favorable characteristics which are particularly surprising, including one or more of enhanced storage stability, high water content (in certain cases, more than 50% up to about 90% by weight water), high inversion temperature, favorable feel (in certain cases, the compositions which actually exhibit a cooling sensation on the skin), an elegant appearance (shiny and/or smooth) and an ability to be formulated into compositions which are useful for unusual applications.

### FIELD OF THE INVENTION

The present invention relates to novel water-in-oil emulsion compositions which exhibit unexpected high temperature (>50° C.) storage stability and other characteristics in a high water content composition. The present compositions comprise a combination of components as an emulsifier mixture, most preferably, dibehenyl fumarate (Marrix® 222), PEG 1500 dihydroxystearate (Arlacel® P135) and monoethanolamine stearamide (also, stearic monoethanolamide, Monamid® S) in combination, and the mixture may be combined with an oil and water soluble, and optionally, numerous other ingredients or components to produce emulsion compositions. The compositions may be used to formulate numerous compositions, and in particular, pharmaceutical or personal care products, such as cosmetics and dermatological agents. The compositions according to the present invention are particularly useful as additives in dermatological products, including cosmetic compositions. The compositions, as emulsions, exhibit a number of favorable characteristics which are particularly surprising, including one or more of enhanced storage stability, high water content (in certain cases, more than 50% and up to about 90% by weight water), high inversion temperature (>50° C.), favorable feel (in certain cases, the compositions which actually exhibit a cooling sensation on the skin), an elegant appearance (shiny and/or smooth) and an ability to be formulated into compositions which are useful for numerous and unusual applications.

### BACKGROUND OF THE INVENTION

The present inventor has spent a number of years in research devoted to understanding and furthering the development of emulsion chemistry for the personal care market. His work with literally thousands of emulsion formulations over the years and his search for new or improved emulsions which exhibit enhanced characteristics such as high water content in a water-in-oil emulsion gave rise to the present formulations. His recent motivation to develop a water-in-oil emulsion able to accommodate a water soluble “active” ingredient, emulsified inside of an oil layer to maintain the stability of the active against for example, chemical oxidation and other chemical forces, after much work, resulted in the present invention.

### BRIEF DESCRIPTION OF THE PRESENT INVENTION

The present invention relates to emulsion compositions comprising:

a) about 1% to about 10-15% by weight of a C₁₂-C₂₂ dialkyl substituted fumarate, preferably dibehenyl fumarate;

b) about 1% to about 10-15% by weight of a polyethylene glycol (PEG) dihydroxystearate, preferably PEG 1500 dihydroxystearate;

c) about 1% to about 10-15% by weight of an alkyl monoethanolamide, preferably stearic monoethanolamide;

d) about 5% to about 85% by weight of an emollient oil; and

e) about 10% to about 90% by weight water, preferably at least about 50% by weight water within this range;

f) optionally, about 0.05% to about 50% by weight of an additive selected from the group consisting of surfactants, skin and hair conditioning agents, coloring agents, pigments, fragrances, humectants, preservatives, anti-oxidants and oil and water soluble “actives” and medicaments, including vitamins, among numerous additional additives, including deodorant compounds, anti-perspirant compounds, including salts, among others, including uv absorbing compounds;

with the proviso that the weight ratio of any one of said components a, b and c in said emulsion composition is no greater than about 3 times and no less than about ⅓ (0.33) the weight ratio of any other component a, b and c in said emulsion composition.

In preferred aspects according to the present invention, the weight ratio of components a, b and c, above, is such that the weight ratio of any one of such components is no greater than about 2 times and no less than about 0.5 times the weight ratio of any other component a, b or c, more preferably components a, b and c are included in compositions in weight ratios which are about 3:2:3 or alternatively, approximately equal, i.e. (about 1.0:1.0:1.0). In more preferable aspects according to the present invention, emulsion compositions (which include water), comprise at least about 4.5-5% (preferably at least about 8-9% by weight within this range) of a, b and c taken together, at least about 10% by weight of an emollient oil and at least about 50% and up to about 90% by weight water, more preferably at least about 60% by weight water, even more preferably at least about 70-75% by weight water, with the remainder of the composition optionally and preferably comprising at least about 0.5% by weight of at least one additive selected from the group consisting of surfactants, skin and hair conditioning agents, coloring agents, pigments, dispersing agents, fragrances, humectants, preservatives, anti-oxidants and oil and water soluble “actives” and medicaments, including vitamins, among numerous additional additives, including deodorant compounds, anti-perspirant compounds, including salts, among others. In certain preferred aspects according to the present invention, emulsion compositions comprise about 3% by weight dibehenyl fumarate, about 2% by weight PEG 1500 dihydroxystearate and about 3% by weight stearic monoethanolamide.

Compositions comprising a mixture of components a, b and c, as otherwise described hereinabove, with or without the inclusion of an emollient oil and/or water are other aspects of the present invention. In those compositions where an emollient oil and water soluble is excluded, compositions according to the present invention consist essentially of a mixture of dialkyl fumarate, preferably, dibehenyl fumarate (DBF); PEG dihydroxystearate, preferably, PEG 1500 dihydroxystearate (PDHS) and monoethanolamine alkylamide, preferably monoethanolamine stearamide (MEAS); wherein the weight ratio of any one of these three components within the mixture of these components, is no greater than about 3 times and no less than about ⅓ the weight ratio of any of the other components within the mixture. In preferred aspects according to the present invention, the weight ratio of any one of these three components within the mixture of the three components is no greater than about 2.0 times and no less than about 0.5 (½) the weight ratio of any of the other components within the mixture. In other preferred embodiments, each of the three components is included in weight ratios of about 3:2:3 or are alternatively, approximately equal to the other components (about 1.0:1.0:1.0). Optionally, other components may be added to the basic mixture of three components, for example, an emollient oil, in a weight ratio ranging from about 1:3 to about 25:1 (or higher) of the weight ratio of the mixture of the three components (e.g., DBF, PDHS and MEAS) described above (i.e., about 33% to about 90% by weight of the three component mixture and the emollient oil, in combination). One or more additives may also be added to the composition containing the three component mixture alone or in further combination with the emollient oil, with those additives being selected from the group consisting of water, surfactants, skin and hair conditioning agents, coloring agents, pigments, dispersing agents, fragrances, humectants, preservatives, anti-oxidants and oil and water soluble “actives” and medicaments, including vitamins, among numerous additional additives, including deodorant compounds, anti-perspirant compounds, including salts, among others.

Methods of making and using the compositions according to the present invention to provide storage stable emulsions and, in particular, in personal care products to promote storage stability and other characteristics as otherwise described herein, are other aspects of the present invention.

### DETAILED DESCRIPTION OF THE INVENTION

The following terms shall be used to describe the present invention:

The term “effective amount” is used throughout the specification, including the claims, to describe amounts or concentrations of individual components used in compositions according to the present invention for the purpose for which they are included in the present compositions. For example, where an emulsifier component is used, an “effective amount” of an emulsifier is that amount which is effective for emulsifying the composition described. The same is true for the other components which comprise the present invention.

The terms “emulsion” and “water-in-oil emulsion” are used synonymously throughout the specification to describe compositions according to the present invention. An “emulsion” according to the present invention is a cream or lotion which is generally formed by the suspension of a very finely divided liquid, in this case water, in another liquid, in this case, an oil. In the present invention, an emulsion is formed when the water phase is compatibilized in the oil phase, for example, such that the water phase becomes “hidden” within the oil phase. While not being limited by way of theory, it is believed that in at least some of the emulsion compositions according to the present invention, the oil phase produces a liposome- or encapsulation-like structure or a related structure surrounding water and/or the water phase, with the three component mixture of the components DBF, PDHS and MEAS serving to enhance the formation of the liposome-like structure and consequently, the emulsion composition. The term emulsion is used to distinguish the present compositions from compositions which contain at least two distinct phases, i.e., an oil phase and a water phase and is non-limiting.

The term “dialkyl fumarate” is used to describe a compound of the structure:

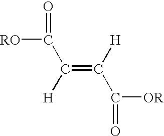

Where R is a C₁₂-C₂₂ straight or branch-chained alkyl group, preferably a C₂₂H₄₅ straight-chained alkyl group, preferably derived from behenyl alcohol, C₂₂H₄₅OH. The preferred dialkyl fumarate for use in the present invention, dibehenyl fumarate (DBF, where R is C₂₂H₄₅), is commercially available from the Bemel Chemical Co., Inc. of Englewood, N.J. DBF functions, at least in part, as a stabilizer in compositions according to the present invention.

The terms “polyethylene glycol dihydroxystearate”, “PEG dihydroxystearate” or “PEG 1500 dihydroxystearate” (also known as polyethylene glycol 1500 dihydroxy stearate or “PDHS”) are used to describe one of the emulsifier components used in the present invention as part of the three component mixture along with the dialkyl fumarate and alkyl monoethanolamide.

These compounds have the general chemical structure:

R′—O(CH₂CH₂—O)_{X}R′

where R′ is a 12-hydroxystearoyl radical (such that R′ is a 12-hydroxy stearoyl group and R′—O form a 12-hydroxystearate ester group as in PEG dihydroxystearate); and x is a positive integer from 16-60 (corresponding to PEG 800 to PEG 3000), preferably 20-50 (corresponding to PEG 1000 to PEG 2500) and most preferably about 28-32, most preferably, PEG 1500). The preferred emulsifiers for use in the present invention include PEG 1500 dihydroxystearate, otherwise known as Arlacel® P135, available from ICI Americas, Inc., Wilmington, Del.). This emulsifier is the reaction product of 12-hydroxy stearic acid and polyethylene glycol of about 1500 average molecular weight.

The terms “alkyl monoethanolamide”, “monoethanolamine alkylamide”, “stearic monoethanolamide” and “monoethanolamine stearamide” are used throughout the specification to describe a third component of the three component mixture which is used in the present invention. This product has the following general structure:

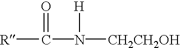

Where R″ is a linear or branch-chained C₁₁-C₂₁ alkyl group, preferably a linear alkyl group, even more preferably, a C₁₇ linear alkyl group (derived from octadecanoic or stearic acid). The preferred alkyl monoethanolamide for use in the present invention is stearic monoethanolamide or monoethanolstearamide, available commercially as as Monamid® S from ICI Americas, Inc., Wilmington, Del. This component functions as an emulsifier in compositions according to the present invention.

The term “oil” or “emollient oil” is used throughout the specification to describe any cosmetically acceptable oil which may be used in personal care products. The term is used throughout the specification to describe any of various lubricious, hydrophobic and combustible substances obtained from animal, vegetable and mineral matter, but is used preferably to describe oils which are derived from vegetable (i.e., non-animal) sources which may be included to embellish the compositions according to the present invention or lower the cost of certain compositions according to the present invention. Emollient oils for use in the present invention may include petroleum-based oil derivatives such as purified petrolatum and mineral oil. Included within this term are the relatively non-polar petrolatum or mineral oil or its derivatives which are hydrocarbons and the synthetic oils, such as esters, which may be referred to as “polar” oils. Preferred oils for use in the present invention include the more hydrophobic mineral oil and petrolatum as well as the more polar esters of, for example, a number of maleates, neopentanoates, citrates, palmitates, stearates, myristates, isostearates and fumarates (either than dibehenyl fumarate, which is used as a stabilizer in the present invention), and any other cosmetically acceptable ester emollient.

Additional oils for use in the present invention may include, for example, mono-, di- and tri-glycerides which may be natural or synthetic (derived from esterification of glycerol and at least one organic acid, saturated or unsaturated, such as for example, butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others, but these are less preferred. In addition, a number of other oils may be used, including, for example, C₁₂ to C₃₀ (or higher) fatty esters or any other acceptable cosmetic emollient.

Typically, the inclusion of for example, isopropyl myristate, octyl palmitate and especially octadodecyl neopentanoate (available from the Bernel Chemical Co., Inc. of Englewood, N.J. as Elefac®I-205), among numerous others, as emollient oils in the present compositions, including personal care products, is also contemplated by the present invention.

The term “storage stability” is used throughout the specification to describe an unexpected characteristic of emulsion compositions according to the present invention which relates to the fact that the present emulsions are generally storage stable at temperatures of at least 50° C. for a period of at least about three months, and often longer than six months, a year or even longer. This is a particularly advantageous feature of emulsion compositions according to the present invention in comparison to prior art compositions, especially given the amount of water which may be used in the present emulsion compositions. Those prior art compositions tend to separate into at least two separate phases, generally a water phase and an oil phase within a relatively short period at a temperature at or above about 50° C. It is quite unexpected, especially given the amount of water which may be included in emulsion compositions according to the present invention, that these compositions would exhibit the degree of storage stability exhibited. It is noted here that all three components of the three component emulsifier mixture must be included in compositions according to the present invention, and in cases where one of the three components is removed from the three component mixture, the resulting compositions lose their ability to maintain storage stability for extended lengths (at least three months) of time as well as certain other unexpected characteristics of the present compositions.

The present invention also relates to a composition consisting essentially of:

a mixture of dialkyl fumarate, preferably, dibehenyl fumarate as component A; PEG dihydroxystearate, preferably, PEG 1500 dihydroxystearate, as component B; and alkyl monoethanolamide, preferably, stearic monoethanolamide, as component C; wherein the weight ratio of any one of said components A, B and C in said composition is no greater than about 3 times and no less than about ⅓ (0.33) the weight ratio of any other component A, B, and C in said composition. In certain preferred aspects according to the present invention, the weight ratio of any one of said components A, B and C in said emulsion composition is no greater than about 2 times and no less than about ½ (0.5) the weight ratio of any other component A, B, and C in said composition. In other preferred compositions according to the present invention, components A, B and C are included in said composition in a weight ratio of about3:2:3 or 1:1:1.

Compositions according to the present invention preferably also contain an emollient oil as previously described herein in amounts ranging from about 1:5 to about 25:1 (or higher) of the weight ratio of the mixture of the three components (the dialkyl fumarate such as DBF, the PEG dihydroxystearate such as PDHS, and the alkyl monoethanolamide, such as MEAS) described above (i.e., about 15-17% to about 90% by weight of the three component mixture and emollient oil, in combination). One or more additives may also be added to the composition containing the three component mixture alone or in further combination with the emollient oil, with those additives being selected from the group consisting of water, surfactants, skin and hair conditioning agents, coloring agents, pigments, dispersing agents, fragrances, humectants, preservatives, anti-oxidants and actives, preferably, oil and water soluble “actives” and medicaments, including vitamins, among numerous additional additives, including deodorant compounds, anti-perspirant compounds, including salts, among others.

The present invention also relates to emulsion compositions comprising:

a) about 1% to about 10-15% by weight dialkyl fumarate, preferably, dibehenyl fumarate;

b) about 1% to about 10-15% by weight PEG dihydroxystearate, preferably, PEG 1500 dihydroxystearate;

c) about 1% to about 10-15% by weight alkyl monoethanolamide, preferably, stearic monoethanolamide;

d) about 5% to about 85% by weight of an emollient oil; and

e) about 10% to about 90% by weight water;

f) optionally, about 0.05% to about 50% by weight of an additive selected from the group consisting of surfactants, skin and hair conditioning agents, coloring agents, pigments, fragrances, humectants, preservatives, anti-oxidants and oil and water soluble “actives” and medicaments, including vitamins, among numerous additional additives, including deodorant compounds, anti-perspirant compounds, including salts, among others; with the proviso that the weight ratio of any one of said components a, b and c in said emulsion composition is no greater than about 3 times and no less than about ⅓ (0.33) the weight ratio of any other component a, b, and c in said emulsion composition.

In preferred aspects according to the present invention the weight ratio of components a, b and c in the emulsion compositions is such that the weight ratio of any one of such components is no greater than about 2 times and no less than about 0.5 times the weight ratio of any of such components, more preferably components a, b and c are included in compositions in weight ratios which are approximately equal, i.e. (about 1.0:1.0:1.0). Alternatively, the three components a, b and c may be included in emulsion compositions preferably in a weight ratio of about 3.0:2.0:3.0. In more preferable aspects according to the present invention, emulsion compositions (which include water) comprise at least about 4.5-5% (preferably at least about 8-9% by weight within this range) of a mixture of components a, b and c, at least about 10% by weight of an emollient oil and at least about 50% by weight water, more preferably at least about 60% by weight water, even more preferably at least about 70-75% by weight water, with the remainder of the composition optionally comprising at least about 0.5% by weight of an additive selected from the group consisting of surfactants, skin and hair conditioning agents, coloring agents, pigments, dispersing agents, fragrances, humectants, preservatives, anti-oxidants and oil and water soluble “actives” and medicaments, including vitamins, among numerous additional additives, including deodorant compounds, anti-perspirant compounds, including salts, among others. In certain preferred aspects according to the present invention, emollient compositions comprise about 3% by weight dibehenyl fumarate, about 2% by weight PEG 1500 dihydroxystearate and about 3% by weight stearic monoethanolamide.

Methods of making and using the compositions according to the present invention to provide emulsion compositions exhibiting enhanced storage stability characteristics and, in particular, in personal care products, are other aspects of the present invention. Personal care products and related formulations are other aspects of the present invention.

In addition to a mixture of the three components (DBF, PDHS and MEAS), an oil and water soluble, emulsion compositions compositions according to the present invention may also comprise, in amounts totalling up to about 50% by weight or more of the final emulsion composition, preferably about 0.5% to about 25% by weight, depending upon the final personal care product to be formulated, one or more optional additive selected from among one or more additional surfactants, fragrances, preservatives, thickeners, gelling agents, anti-oxidants, vitamins, pigments, coloring agents, dispersing agents, conditioning agents, uv absorbing agents and humectants, among numerous other standard cosmetic additives, as herein described.

In general, compositions according to the present invention are included in end-use formulations (personal care products) in amounts ranging from less than about 1% to about 99% by weight or more, preferably within the range of at least about 10% by weight to about 75% by weight or more, depending upon the end-use of those compositions.

For example, the present compositions may be utilized as moisturizing barrier (water repellent) creams, lotions or emulsions for use in hair dressings and creams, skin care products including moisturing lotions and creams, hand creams/lotions, color cosmetics, sunscreens, skin protective compositions, vitamin or other active creams/lotions and any skin care product where delivery is through or by way of an emulsion.

Compositions according to the present invention may be generally made by mixing the individual components together, in any order, at elevated temperature, i.e., preferably at a temperature of at least about 75-85° C. Emulsion compositions according to the present invention may be made by mixing the individual components in any order at elevated temperature, but are preferably made by first preparing a mixture of the dialkyl fumarate, the PEG dihydroxystearate and the alkylmonoethanolamide (e.g., DBF, PDHS and MEAS) at elevated temperature (preferably, above about 75° C., more preferably above about 85° C.) separately, then combining an oil with the three component mixture, and then adding water and mixing at elevated temperatures. In alternative embodiments, compositions according to the present invention may be formulated as a mixture of an oil and the three component mixture of e.g, DBV, PDHS and MEAS and further additives, including water may be mixed into final compositions thereafter as a mixture of an oil phase with a water phase, which mixing also occurs at an elevated temperature. Generally, the temperature at which mixing is effected is preferably at least about 50° C., more preferably at least about 65 to 75° C., even more preferably at least about 75 to 85° C., and most preferably at least about 80-85° C. The foregoing are temperatures which are generally effective to allow the oil phase to remain soluble within itself (at a temperature wherein the oil phase remains clear and in a solution) during mixing of the emulsion compositions according to the present invention. After mixing for at least about 10-15 minutes, more preferably at least 30 minutes or more (depending upon batch size) at elevated temperatures, the mixture is then cooled before use and/or packaging. Mixing may be performed in a simple propeller mixer with vortex formation without the application of high shear force, although in certain cases high shear force may be desirable. Although one could use higher mixing speeds, because of the ease of mixing the present compositon, this approach is not generally necessary.

As discussed, all components may be mixed together in a one pot preparation, or one or more components (such as the oil phase, water phase and/or other additives) may be prepared separately and then combined. In preferred embodiments, after the oil phase (containing the three component mixture and an emollient oil) is first prepared, the oil phase is added to the water phase and the combined phases are mixed thoroughly for maximum result. It is noted that the preferred method for making the present composition comprises first making the oil phase, making the water phase separately (depending upon the embodiment, the water phase may contain only water or water and other water soluble/immiscible agents), preferably adding the water phase to the oil phase, followed by mixing the phases together, all at elevated temperature. Alternatively, it is possible to separately mix the individual components in a single pot preparation, or to mix the individual components in a different order.

Having generally described the invention, reference is now made to the following examples which are intended to illustrate preferred embodiments and comparisons. The included examples are not to be construed as limiting the scope of this invention as is more broadly set forth above and in the appended claims.

### EXAMPLES

### Example 1

### Emulsifier Pre-Base

The following components were mixed in a container capable of heating and cooling samples and having a sufficiently large enough capacity to hold the composition. Each of the ingredients is added at room temperature and the mixture is then heated to 85° C. and mixed to homgeneity. After thorough mixing, the mixture is cooled to 70° C. and flaked. The flakes may be packaged and sold as an emulsifier pre-base or used to directly to produce emulsion compositions according to the present invention. Note that the present emulsifier pre-base may be use d in phase A of example 2, below.

| | Component | Weight % |
|---|---|---|
| (heat to 85° C. | Dibehenyl fumarate (Marrix 222 ™) | 33.34 |
| and mix - | PEG 1500 Dihydroxystearate (Arlacel ™ P135) | 33.33 |
| thereafter flake | Monoethanolamine stearamide (Monamid ™ S) | 33.33 |
| at 70° C.) | 100.00% | Total |

### Example 2

### Emulsion Composition

The following components were combined in three separate phases, phase A, the oil phase, phase B, the water phase and phase C, the preservative phase. To make the emulsion, add phase B (the water) to phase A (which may be used as individual components as indicated or as a pre-mix from example 1, 9% of total) at 85° C., then cool to 70° C. and add phase C (preservative). The mixture is allowed to further cool to between 35° and 45° C. and is further mixed to homogeneity at that temperature to avoid aeration within the product. The product is then packaged for sale.

| | Component | Weight % |
|---|---|---|
| Phase A: | | |
| (heat to 85° C. | Dibehenyl fumarate (Marrix 222 ™) | 3.00 |
| and mix) | PEG 1500 Dihydroxystearate (Arlacel ™ P135) | 3.00 |
| | Monoethanolamine stearamide (Monamid ™S) | 3.00 |
| | Octyldodecyl neopentanoate (Elefac ™ I-205) | 11.00 |
| Phase B: | Water, deionized | 79.90 |
| (Heat to 85° C.) | | |
| Phase C: | Preservative (Kathon ™ CG) | 0.10 |
| (Add to phases A and B | 100.00% | |
| at 70° C, cool to 35-45° C. | | |
| and mix) | | |

Comments:

1. This is a water-in-oil emulsion which can be used for further formulation of personal care products.

It is to be understood by those skilled in the art that the foregoing description and examples are illustrative of practicing the present invention, but are in no way limiting. Variations of the detail presented herein may be made without departing from the spirit and scope of the present invention as defined by the following claims.

1. A composition consisting essentially of a mixture of:
a). dialkyl fumarate according to the structure: 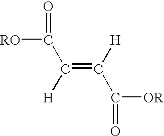
where R is a C₁₂-C₂₂ linear or branch-chained alkyl group, as component A of said mixture;
b). a polyethyleneglycol dihydroxystearate according to the structure:
R′—O(CH₂CH₂—O)_{X}R′
where R′ is a 12-hydroxystearoyl group and X is an integer from 16 to 60, as component B of said mixture; and
c). an alkyl monoethanolamide according to the structure: 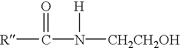
where R″ is a C₁₁-C₂₁ linear or branch-chained alkyl group, as component C of said mixture; wherein the weight ratio of any one of said components A, B and C in said composition is no greater than about 3 times and no less than about ⅓ the weight ratio of any other component A, B, and C in said composition.

2. The composition according to claim 1 wherein said dialkyl fumarate is dibehenyl fumarate, said polyethyleneglycol dihydroxystearate is PEG 1500 dihydroxystearate and said alkyl monoethanolamide is stearic monoethanolamide.

3. The composition according to claim 1 wherein the weight ratio of any one of said components A, B and C in said composition is no greater than about 2 times and no less than about ½ the weight ratio of any other component A, B, and C in said composition.

4. The composition according to claim 2 wherein the weight ratio of components A, B and C in said composition is about 1:1:1.

5. The composition according to claim 2 wherein the weight ratio of components A, B and C in said composition is about 3:2:3.

6. The composition according to claim 1 further including an effective amount of at least one additive selected from the group consisting of surfactants, fragrances, preservatives, thickeners, gelling agents, anti-oxidants, vitamins, pigments, coloring agents, dispersing agents, conditioning agents, uv absorbing agents and humectants.

7. The composition according to claim 2 further including an effective amount of at least one additive selected from the group consisting of surfactants, fragrances, preservatives, thickeners, gelling agents, anti-oxidants, vitamins, pigments, coloring agents, dispersing agents, conditioning agents, uv absorbing agents and humectants.

8. A composition in the form of an emulsion comprising:
a) about 1% to about 15% by weight of a dialkyl fumarate according to the structure: 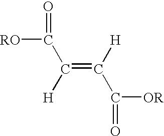
where R is a C₁₂-C₂₂ linear or branch-chained alkyl group as component A of said mixture;
b) about 1% to about 15% by weight of a polyethyleneglycol dihydroxystearate according to the structure:
R′—O(CH₂CH₂—O)_{X}R′
where R′ is a 12-hydroxystearoyl group and X is an integer from 16 to 60, as component B of said mixture;
c) about 1% to about 15% by weight of an alkyl monoethanolamide according to the structure: 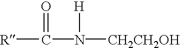
where R″ is a C₁₁-C₂₁ linear or branch-chained alkyl group as component C of said mixture;
d) about 5% to about 85% by weight of an emollient oil;
e) about 10% to about 90% by weight water; and
f) optionally, about 0.05% to about 50% by weight of an additive selected from the group consisting of surfactants, conditioning agents, coloring agents, pigments, fragrances, humectants, preservatives, anti-oxidants and oil and water soluble “actives” and medicaments, including vitamins, among numerous additional additives, including deodorant compounds, antiperspirant compounds, including salts, among others; with the proviso that the weight ratio of any one of said components A, B and C in said emulsion composition is no greater than about 3 times and no less than about ⅓ the weight ratio of any other component A, B and C in said emulsion composition.

9. The composition according to claim 8 wherein said dialkyl fumarate is dibehenyl fumarate, said polyethyleneglycol dihydroxyalkanoate is PEG 1500 dihydroxystearate and said alkyl monoethanolamide is stearic monoethanolamide.

10. The composition according to claim 8 wherein the weight ratio of any one of said components A, B and C in said composition is no greater than about 2 times and no less than about ½ the weight ratio of any other component A, B and C in said composition.

11. The composition according to claim 9 wherein the weight ratio of said components A, B and C in said composition is about 1:1:1.

12. The composition according to claim 9 wherein the weight ratio of said components A, B and C in said composition is about 3:2:3.

13. The composition according to claim 8 wherein said emollient oil is selected from the group consisting of petrolatum and mineral oil, fatty esters of maleates, neopentanoates, citrates, fumarates, esters of myristates, stearates, isostearates, palmitates, and animal and vegetable glyceride esters.

14. The composition according to claim 8 wherein said emollient oil is an animal or vegetable oil selected from the group consisting of soybean, sunflower, safflower, cottonseed, linseed, iticica, tung, castor, cocoanut, palm oil, fish oil and fish liver oil.

15. The composition according to claim 9 wherein said emollient oil is selected from the group consisting of petrolatum and mineral oil, fatty esters of maleates, neopentanoates, neopentanoyls, citrates, fumarates, esters of myristates, stearates, isostearates, palmitates, and animal and vegetable glyceride esters.

16. The composition according to claim 9 wherein said emollient oil is an animal or vegetable oil selected from the group consisting of soybean, sunflower, safflower, cottonseed, linseed, iticica, tung, castor, cocoanut, palm oil, fish oil and fish liver oil.

17. The composition according to claim 11 wherein said emollient oil is selected from the group consisting of petrolatum and mineral oil, fatty esters of maleates, neopentanoates, citrates, fumarates, esters of myristates, stearates, isostearates, palmitates, and animal and vegetable glyceride esters.

18. The composition according to claim 11 wherein said emollient oil is an animal or vegetable oil selected from the group consisting of soybean, sunflower, safflower, cottonseed, linseed, iticica, tung, castor, cocoanut, palm oil, fish oil and fish liver oil.

19. The composition according to claim 9 wherein said emollient oil is selected from the group consisting of octyldodecyl neopentanoate, isopropyl myristate and octyl palmitate.

20. The composition according to claim 11 wherein said emollient oil is selected from the group consisting of octyldodecyl neopentanoate, isopropyl myristate and octyl palmitate.

21. The composition according to claim 8 wherein said water comprises at least about 50% by weight of said composition.

22. The composition according to claim 9 wherein said water comprises at least about 50% by weight of said composition.

23. The composition according to claim 10 wherein said water comprises at least about 50% by weight of said composition.

24. The composition according to claim 10 wherein said water comprises at least about 50% by weight of said composition.

25. A personal care composition in the form of an emulsion comprising:
a) about 1% to about 15% by weight of a dialkyl fumarate according to the structure: 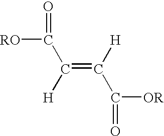
where R is a C₁₂-C₂₂ linear or branch-chained alkyl group as component A of said mixture;
b) about 1% to about 15% by weight of a polyethyleneglycol dihydroxystearate according to the structure:
R′—O(CH₂CH₂—O)_{X}R′
where R′ is a 12-hydroxystearoyl group and X is an integer from 16 to 60, as component B of said mixture;
c) about 1% to about 15% by weight of an alkyl monoethanolamide according to the structure: 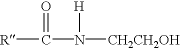
where R″ is a C₁₁-C₂₁ linear or branch-chained alkyl group, as component C of said mixture;
d) about 5% to about 85% by weight of an emollient oil;
e) about 10% to about 90% by weight water; and
f) about 0.5% to about 50% by weight of at least one additive selected from the group consisting of surfactants, skin and hair conditioning agents, coloring agents, pigments, fragrances, humectants, preservatives, anti-oxidants, oil and water soluble actives and medicaments, deodorant compounds, antiperspirant compounds, and mixtures thereof, with the proviso that the weight ratio of any one of said components A, B and C in said emulsion composition is no greater than about 3 times and no less than about ⅓ the weight ratio of any other component A, B and C in said emulsion composition.

26. The composition according to claim 25 wherein said dialkyl fumarate is dibehenyl fumarate, said polyethyleneglycol dihydroxystearate is PEG 1500 dihydroxystearate and said alkyl monoethanolamide is stearic monoethanolamide.

27. The composition according to claim 25 wherein the weight ratio of any one of said components A, B and C in said composition is no greater than about 2 times and no less than about ½ the weight ratio of any other component A, B and C in said composition.

28. The composition according to claim 26 wherein the weight ratio of said components A, B and C in said composition is about 1:1:1.

29. The composition according to claim 26 wherein the weight ratio of said components A, B and C in said composition is about 3:2:3.

30. The composition according to claim 26 wherein said emollient oil is selected from the group consisting of petrolatum and mineral oil, fatty esters of maleates, neopentanoates, citrates, fumarates, esters of myristates, stearates, isostearates, palmitates, and animal and vegetable glyceride esters.

31. The composition according to claim 26 wherein said emollient oil is an animal or vegetable oil selected from the group consisting of soybean, sunflower, safflower, cottonseed, linseed, iticica, tung, castor, cocoanut, palm oil, fish oil and fish liver oil.

32. The composition according to claim 25 comprising at least 50% by weight water.

33. The composition according to claim 26 comprising at least 50% by weight water.

34. The composition according to claim 27 comprising at least 50% by weight water.

35. The composition according to claim 28 comprising at least 50% by weight water.

36. The composition according to claim 29 comprising at least 50% by weight water.
